(19) United States
(12) Patent Application Publication
(10) Pub. No.: US 2020/0297820 A1
(43) Pub. Date: Sep. 24, 2020

(54) STIMULATION OF HUMAN MEIBOMIAN GLAND FUNCTION

(71) Applicant: The Schepens Eye Research Institute, Inc., Boston, MA (US)

(72) Inventors: David A. Sullivan, Boston, MA (US); Juan Ding, Boston, MA (US); Yang Liu, Boston, MA (US); Wendy Kam, Boston, MA (US)

(21) Appl. No.: 16/694,494

(22) Filed: Nov. 25, 2019

Related U.S. Application Data
(63) Continuation of application No. 14/546,612, filed on Nov. 18, 2014, now abandoned.
(60) Provisional application No. 61/905,613, filed on Nov. 18, 2013.

(51) Int. Cl. A61K 38/30; A61K 31/5685; A61K 31/7052; A61K 38/28; A61K 38/27; A61K 47/36; A61K 31/573; A61K 45/06; A61K 31/568; A61K 9/00; A61K 31/57

(52) U.S. Cl. CPC A61K 38/30; A61K 31/5685; A61K 31/7052; A61K 38/28; A61K 38/27; A61K 31/57; A61K 31/573; A61K 45/06; A61K 31/568; A61K 9/0048; A61K 47/36

(57) ABSTRACT

Disclosed herein are systems and methods for stimulating meibomian gland epithelial cell function by administering to the ocular surface or immediate vicinity of an eye of a subject an effective amount of a pharmaceutical composition containing a PLD-inducing compound, such as the cationic amphiphilic drugs (e.g. azithromycin), androgen or an androgen analogue with androgen effectiveness, corticosteroid, progesterone, IGF-1 or an IGF-1 analogue (e.g. insulin), GH, and mixtures thereof. The pharmaceutical compositions are effective to treat a variety of aliments to the eye including meibomian gland dysfunction, evaporative dry eye disease, lipid abnormalities in meibum or the tear film, and autoimmune diseases such as Sjögren's syndrome.

### PRIORITY CLAIM

This Application is a continuation of U.S. application Ser. No. 14/546,612, filed Nov. 18, 2014, which claims priority to U.S. Provisional Application No. 61/905,613, filed 18 Nov. 2013, the contents of which are herein incorporated by reference in their entirety.

### TECHNICAL FIELD

This disclosure relates to compositions for the stimulation of meibomian gland epithelial cell function in humans. More particularly, the disclosure is directed to the use of one or more therapeutic agents for treating diseases of the eye including meibomian gland dysfunction (MGD), evaporative dry eye disease, lipid abnormalities of meibum or the tear film, and autoimmune diseases such as Sjögren's syndrome. The preferred therapeutic agents include phospholipidosis (PLD)-inducing compounds, such as the cationic amphiphilic drugs (e.g. azithromycin), androgen or an androgen analogue, corticosteroid, progesterone, insulin-like growth factor 1 (IGF-1) or an IGF-1 analogue (e.g. insulin), growth hormone (GH), and mixtures thereof.

### BACKGROUND

The meibomian glands are sebaceous glands located in the upper and lower eyelids. The meibomian glands are responsible for the supply of meibum to the tear film. Meibum is a lipid and protein mixture that provides a clear optical surface for the cornea, interferes with bacterial colonization, retards tear overflow, promotes the stability and prevents the evaporation of the tear film. The major cause of dry eye disease (DED) is obstructive MGD.

The tear film plays an essential role in maintaining the integrity of the ocular surface, protecting against microbial challenge, and preserving visual acuity. These functions are dependent upon the composition and stability of the tear film structure, which includes an underlying mucin foundation (goblet cells, conjunctival epithelial cells, and corneal epithelial cells), a middle aqueous component (lacrimal gland epithelial cells), and an overlying lipid layer (secreted by meibomian gland epithelial cells). The disruption, deficiency or absence of the tear film may impact the eye by leading to increased shear stress on the ocular surface, the desiccation of the corneal epithelium, the ulceration and perforation of the cornea, a greater susceptibility to infectious disease, and ultimately, pronounced visual impairment and blindness.

DED afflicts millions of individuals and is one of the most frequent causes of patient visits to eye care practitioners. Dry eye is characterized by a cycle of tear film hyperosmolarity and ocular surface inflammation, leading to increased friction and damage to the eye. The impact of moderate to severe dry eye disease is associated with significant pain, low vitality, and poor general health. The burden of dry eye disease for the US healthcare system is estimated to be in the billions of dollars.

MGD, stemming from hyperkeratinization of the ductal epithelium and reduced meibum output/quality, destabilizes the tear film, and increases evaporation and osmolarity. MGD frequently leads to cystic dilation of glandular ducts, acinar cell atrophy, loss of secretory epithelial cells (meibocytes), and lipid insufficiency. MGD may also facilitate bacterial growth on the lid margin and promote inflammation in the adjacent conjunctiva (posterior blepharitis).

Antibiotics have been used in the past to manage MGD, or the symptoms of MGD, and can act as an anti-inflammatory to suppress MGD-associated posterior blepharitis and the growth of eyelid bacteria. However, these antibiotics have not been shown to act directly on human meibomian gland dysfunction.

Various approaches to address MGD have been proposed. See, for instance, U.S. Published Patent Application No. US 2012/0003296, to Shantha et al., speculate to the use of insulin, and/or IGF-1 analogues eye drops for treating dry eye syndrome due to etiological factors, including Sjögren's syndrome, and glandular malfunction in the eyelids, including MGD. Shantha et al. provide no data to support their speculation. Foulks et al., Cornea, 29(7), 781-788 (2010), describes various proposed treatments for the signs and symptoms of MGD, using topical therapy with azithromycin. Foulks et al. could not detect any azithromycin in meibum. They speculate that the “improvement in the signs of redness and swelling of the eyelid margin could result from the antibacterial effect of azithromycin reducing bacterial presence or to the known anti-inflammatory properties of azithromycin.” They also speculate that the “improvement of the degree of meibomian gland orifice plugging and the character of meibomian gland secretion is, however, more likely due to a physical change in the meibum. The mechanism of action of azithromycin on meibomian gland lipid may be related to inhibition of tissue or bacterial lipases that otherwise degrade the lipid structure.” They concluded that “Further studies to clarify the mechanism of action are needed.” Foulks et al provide no data to show that azithromycin acts directly on human meibomian gland epithelial cells.

In spite of the efforts expended in searching for a safe and effective treatment for MGD, there is no global cure for MGD and its evaporative DED.

In view of the aforementioned, a clinical need exists to develop a safe and effective treatment for the stimulation of human meibomian gland epithelial cell function. This and other objectives will be clear from the following description.

### SUMMARY

Disclosed herein are methods and systems for the stimulation of human meibomian gland epithelial cell function. In particular, the methods involve treating the meibomian gland with an effective amount of a pharmaceutical composition containing a treatment agent selected from the group consisting of PLD-inducing compounds, such as the cationic amphiphilic drugs (e.g. azithromycin), androgen or an androgen analogue, corticosteroid, progesterone, IGF-1, IGF-1 analogue (e.g. insulin), GH, and mixtures thereof, for a period of time sufficient to effect the stimulatory action.

In one embodiment, the amount of treatment agent used in the pharmaceutical composition is as follows: 2% by mass/volume (i.e. g/100 ml) or less of PLD-inducing compound, such as a cationic amphiphilic drug (e.g. azithromycin), 0.2% by mass/volume of androgen or an androgen analogue that has androgen effectiveness, 1% by mass/volume or less of corticosteroid, 1% by mass/volume or less of progesterone, 1 μM or less of IGF-1, IGF-1 analogues, or 50 nM or less of growth hormone (GH), and mixtures thereof.

In another embodiment, the androgen analogue is selected from the group consisting of 17α-methyl-17β-hydroxy-2-oxa-5α-androstan-3-one, 17β-hydroxy-5α-androstane derivative containing a ring A unsaturation, a testosterone derivative, a 4,5α-dihydrotestosterone derivative, a 19-nortestosterone derivative and a nitrogen-substituted androgen.

In a further embodiment, the pharmaceutical composition can include a hyaluronate, an electrolyte, an ophthalmic demulcent, an excipient, an astringent, a vasoconstrictor and/or an emollient.

In one aspect, the electrolyte is selected from the group consisting of sodium chloride, potassium chloride, sodium bicarbonate, potassium bicarbonate, calcium chloride, magnesium chloride, trisodium citrate, hydrochloric acid, sodium hydroxide, and mixtures thereof.

In a still further embodiment, a method is described of stimulating human meibomian gland epithelial cell function, the method including providing a therapeutic agent including a therapeutically effective amount of PLD-inducing compounds, such as the cationic amphiphilic drugs (e.g. azithromycin), androgen or an androgen analogue that has androgen effectiveness, corticosteroid, progesterone, IGF-1 or an IGF-1 analogue (e.g. insulin), GH, and mixtures thereof topically to the ocular surface or immediate vicinity of an eye of a patient showing signs or symptoms of meibomian gland dysfunction, evaporative dry eye, lipid abnormality of meibum or the tear film and/or Sjögren's syndrome.

The foregoing embodiments and aspects of the disclosure are illustrative only, and are not meant to restrict the spirit and scope of the claims.

### BRIEF DESCRIPTION OF THE DRAWINGS

The foregoing and other advantages and features of the disclosure will become apparent upon reading the following detailed description with reference to the accompanying figures and drawings.

FIG. 1 illustrates a series of photomicrographs showing the effect of azithromycin on the morphology and time-dependent accumulation of lipids in immortalized human meibomian gland epithelial cells (IHMGEC) in serum-containing media for days 1 to 7;

FIG. 2 illustrates a graph showing the effect of azithromycin on the accumulation of lipids in IHMGEC for days 1 to 7 compared to a control;

FIG. 3 illustrates a series of photomicrographs showing the cellular morphology of IHMGEC treated with azithromycin compared to a control for days 1 to 7;

FIG. 4 illustrates a graph showing the effect of azithromycin on immortalized human meibomian gland epithelial cell proliferation in the absence of serum for days 1 to 7; and

FIG. 5 illustrates a graph showing the effect of azithromycin on immortalized human meibomian gland epithelial cell proliferation in the presence of serum for days 1 to 7;

FIGS. 6a, 6b, and 6c illustrate the effect of AZM on intracellular accumulation of lipids and lysosomes;

FIG. 7 illustrates the influence of AZM on the accumulation of lysosomal lamellar bodies in human meibomian gland epithelial cells;

FIG. 8a illustrates the effect of AZM on the expression of cholesterol ester in cells treated with vehicle or 10 micrograms/milliliter AZM;

FIG. 8b illustrates band intensity of the cells shown in FIG. 8a;

FIGS. 9a, 9b, and 9c illustrate the effects of insulin-like growth factor 1 (IGF-1) and growth hormone (GH) on signaling pathways in human meibomian gland epithelial cells;

FIGS. 10a, 10b, and 10c illustrate the influence of IGF-1 on the proliferation of human meibomian gland epithelial cells;

FIGS. 11a, 11b, 11c, and 11d illustrate the effect of IGF-1 on sterol regulatory element-binding protein (SREBP-1) expression and lipid accumulation in human meibomian gland epithelial cells;

FIGS. 12a and 12b illustrate the inhibition of IGF-1 action by an IGF-1 receptor (IGF-1R)-blocking antibody in human meibomian gland epithelial cells;

FIGS. 13a, 13b, and 13c illustrate the effect of IGF-1, AZM, and IFG-1+AZM combination on intracellular accumulation of lipids and lysosomes;

FIGS. 14a and 14b illustrate the effect of IGF-1, AZM, and IFG-1+AZM combination on the accumulation of CE, TG, FC, PE, and PC;

FIGS. 15a, 15b, 15c, and 15d illustrate the effect of IGF-1, AZM, and IFG-1+AZM combination on the expression of SREBP-1, cyclins B1, and D1;

FIG. 16 illustrates the effect of IGF-1, AZM, and IFG-1+AZM combination on the proliferation of IHMGECs;

FIG. 17a illustrates how insulin activates AKT signaling in a dose-dependent manner;

FIG. 17b illustrates that insulin activation of AKT is similarly regulated by an IGF-1 receptor blocking antibody;

FIG. 17c illustrates that IGF-1 receptor antibody diminishes the IGF-1 receptor without affecting the insulin receptor;

FIG. 18 illustrates that insulin promotes human meibomian gland epithelial cell proliferation;

FIG. 19 illustrates that insulin promotes human meibomian gland epithelial cell accumulation of neutral lipids;

FIG. 20 illustrates increased meibomian gland size in bGH mice;

FIG. 21 illustrates decreased meibomian gland size in GHR-/- mice that have no growth hormone receptors;

FIG. 22 illustrates decreased meibomian gland size in GHA mice which have growth hormone deficiency; and

FIG. 23 illustrates relative meibomian gland size when normalized to the WT controls for GHR-/-, GHA, and bGH mice.

### DETAILED DESCRIPTION

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All technical and patent publications cited herein are incorporated herein by reference in their entirety.

The practice of the present disclosure will be the application if a therapeutic agent including a therapeutically effective amount of azithromycin, androgen or an androgen analogue that has androgen effectiveness, corticosteroid, progesterone, IGF-1 or an IGF-1 analogue (e.g. insulin), GH, and mixtures thereof topically to the ocular surface or immediate vicinity of an eye

All numerical designations, e.g., pH, temperature, time, concentration, and molecular weight, including ranges, are approximations which are varied (+) or (−) by increments of 1.0 or 0.1, as appropriate. It is to be understood, although not always explicitly stated, that all numerical designations are preceded by the term “about”. It also is to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein also encompass any and all possible subranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above.

As used in the specification and claims, the singular form “a”, “an” and “the” include plural references unless the context clearly dictates otherwise. For example, the term “a pharmaceutically acceptable carrier” includes a plurality of pharmaceutically acceptable carriers, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transitional terms are within the scope of this disclosure.

As used herein, the terms “treating,” “treatment” and the like are used herein to mean obtaining a desired effect or stimulating human meibomian gland epithelial cell function.

A “composition” is intended to mean a combination of active agent and another compound or composition, inert (for example, a detectable agent or label) or active. A “pharmaceutical composition” is intended to include the combination of an active agent with a carrier, making the composition suitable for diagnostic or therapeutic use in vitro, in vivo or ex vivo.

The term “pharmaceutically acceptable carrier”, which may be used interchangeably with the term biologically compatible carrier, refers to reagents, compounds, materials, compositions, and/or dosage forms that are not only compatible with the other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use include, but are not limited to, liquids and semi-solids (e.g., gels).

An “effective amount” is an amount sufficient to effect beneficial or desired results. An effective amount can be administered in one or more administrations, applications or dosages.

As used herein, the term “administering” means providing the subject with an effective amount of the composition effective to achieve the desired object of the method. Methods of administering composition such as those described herein are well known to those of skill in the art and include, but are not limited to topical or local administration. Administration can be effected continuously or intermittently throughout the course of treatment. Methods of determining the most effective means and dosage of administration are well known to those of skill in the art and will vary composition used for therapy and the subject being treated. Single or multiple administrations can be carried out with the dose level and pattern being selected by the treating clinician. The term “MGD” as used herein refers to meibomian gland dysfunction, a leading cause of dry eye disease, herein “DED”.

Disclosed herein are compositions for the stimulation of meibomian gland epithelial cell function in humans. More particularly, use of one or more therapeutic agents for stimulating meibomian gland epithelial cell function, wherein the therapeutic agents include PLD-inducing compounds, such as the cationic amphiphilic drugs (e.g. azithromycin), androgen or an androgen analogue, corticosteroid, progesterone, IGF-1 or an IGF-1 analogue (e.g. insulin), GH, and mixtures thereof are disclosed herein.

The amount of each therapeutic agent required depends on the particular agent. In general, the following amounts have been found to be particularly effective: 2% by mass/volume or less of a PLD-inducing compound, such as a cationic amphiphilic drug (e.g. azithromycin), 0.2% by mass/volume of androgen or an androgen analogue, 1% by mass/volume or less of corticosteroid, 1% by mass/volume or less of progesterone, 1 μM or less of IGF-1 or an IGF-1 analogue (e.g., insulin), or 50 nM or less of GH, and mixtures thereof.

In a preferred embodiment, the androgen analogue can advantageously be one or more of the following specific or subgeneric analogues: 17α-methyl-17β-hydroxy-2-oxa-5α-androstan-3-one, 17β-hydroxy-5α-androstane derivative containing a ring A unsaturation, a testosterone derivative, a 4,5α-dihydrotestosterone derivative, a 19-nortestosterone derivative and a nitrogen-substituted androgen.

The pharmaceutical composition includes one or more of the therapeutic agents identified above and a pharmaceutically acceptable carrier or adjuvant. This can include a hyaluronate (or hyaluronic acid), an electrolyte, an ophthalmic demulcent, an excipient, an astringent, a vasoconstrictor and/or an emollient. Preferred electrolytes include, for example, sodium chloride, potassium chloride, sodium bicarbonate, potassium bicarbonate, calcium chloride, magnesium chloride, trisodium citrate, hydrochloric acid, sodium hydroxide, and mixtures thereof. The amounts of electrolytes used can vary, but preferred electrolyte ranges are as follows (in mole fractions): 44% to 54% of sodium chloride, 8% to 14% of potassium chloride, 8% to 18% of sodium bicarbonate, 0% to 4% of potassium bicarbonate, 0% to 4% of calcium chloride, 0% to 4% of magnesium chloride, 0% to 4% of trisodium citrate, 0% to 20% of hydrochloric acid, and 0% to 20% of sodium hydroxide.

In order to identify subjects who would benefit from the stimulation of meibomian gland epithelial cell function, it is useful to identify the symptoms of an associated disorder of the ocular surface. The following eye disorders have been found to have such an association: meibomian gland dysfunction (MGD), evaporative DED, lipid abnormalities of meibum or the tear film, and autoimmune disorders such as Sjögren's syndrome.

As noted, MGD is believed to be the most common cause of DED. Typically, the meibomian gland produces and releases a lipid and protein mixture that promotes the stability and prevents the evaporation of the tear film, thereby playing an essential role in ocular surface health. Conversely, MGD destabilizes the tear film and increases its evaporation. MGD is caused primarily by hyperkeratinization of the terminal duct epithelium and reduced secretion quality, and can lead to cystic dilation of glandular ducts, acinar cell death and lipid deficiency. The end result is evaporative DED, characterized by a cycle of tear film hyperosmolarity and ocular surface stress, and leading to increased friction, inflammation and damage to the eye. Moderate to severe DED is associated with significant pain, role limitations, low vitality and poor general health.

The therapeutic agents possess anti-inflammatory and/or anti-bacterial characteristics. However, these agents have not been shown to act directly on human meibomian gland epithelial cells to enhance function and to ameliorate the pathophysiology of MGD. While not wishing to be bound by any particular theory, the therapeutic agents act directly on the human meibomian gland epithelial cells to stimulate their function. This activity serves to enhance the quality and quantity of lipid production, and promote holocrine secretion.

The disclosure may be further described and illustrated in the following examples which are not intended to limit the scope of the remaining portions of the disclosure or the claims.

### EXAMPLES

A series of experiments with immortalized human meibomian gland epithelial cells led to the following discoveries:

Azithromycin (10 μg/ml) induces a striking, time-dependent accumulation of lipid in immortalized human meibomian gland epithelial cells. Within 3 days of azithromycin exposure, the number, size and staining intensity of intracellular lipid-containing vesicles markedly increases, as compared to those of vehicle-treated control cells. This azithromycin effect on lipids appears to become maximal after 5 to 7 days of treatment, and is associated with an increased expression of sterol regulatory element binding protein 1 (SREBP-1) protein. SREBP-1 is a transcription factor that regulates the genes required for de novo lipogenesis.

Azithromycin appears to promote terminal maturation of immortalized human meibomian gland epithelial cells, as well as their holocrine-like secretion.

Dihydrotestosterone (an androgen), dexamethasone (a glucocorticoid) and progesterone (a progestin), at physiological concentrations of 10⁻⁸ M, each promote marked lipid accumulation in immortalized human meibomian gland epithelial cells. These effects occur within 5 to 7 days of treatment.

IGF-1 (10 nM), GH (10 nM), IGF-1 plus GH, or individually or both in combination with DHT (10 nM), cause a significant accumulation of lipid in immortalized human meibomian gland epithelial cells. This effect is evident by 3 days of treatment, and continues to increase at least up to 13 days of hormone exposure. The IGF-1 actions, either alone or with GH and/or DHT, are associated with a significant increase in the expression of SREBP-1 protein.

IGF-1 increases the proliferation of immortalized human meibomian gland epithelial cells. This effect is quite notable because meibomian gland secretion is critically dependent upon the active proliferation of glandular epithelial cells. Once generated, these sebaceous-like cells undergo a maturation process towards lipid accumulation, terminal differentiation and holocrine secretion. Such secretion involves the death and disintegration of fully mature, lipid-rich epithelial cells, their release into glandular ductules, and ultimately, delivery to the ocular surface. Given this continual loss of cells, stimulation of epithelial cell proliferation is extremely important and promotes not only meibocyte replenishment, but also the production of meibum.

IGF-1 increases the phosphorylation of Akt in immortalized human meibomian gland epithelial cells. This Akt action, which occurs within minutes, may be a primary mechanism by which IGF-1 promotes proliferation of these cells.

IGF-1, GH and DHT increase the phosphorylation of extracellular-signal-regulated kinases (ERKs), which may mediate, at least in part, the hormonal regulation of cell proliferation and post-mitotic, differentiated functions.

IGF-1 attenuates the 13-cis RA-induced late apoptosis/necrosis of immortalized human meibomian gland cells. This 13-cis RA effect appears to be one mechanism by which this retinoic acid derivative causes MGD.

As an example of these experiments, immortalized human meibomian gland epithelial cells were cultured in the presence or absence of 10% fetal bovine serum. Cells were treated with an ethanol vehicle or azithromycin (10 μg/ml; Santa Cruz Biotechnology) for varying time periods. Cellular morphological appearance was recorded, cells were counted with a hemocytometer, and lipid accumulation was assessed by staining the cells with LipidTOX green neutral lipid stain (Invitrogen, Grand Island, N.Y.), according to reported methods. Staining fluorescent intensities were quantified by using ImageJ (http://rsbweb.nih.gov/ij/index.html). Statistical analyses were performed with Student's t-test (two-tailed, unpaired).

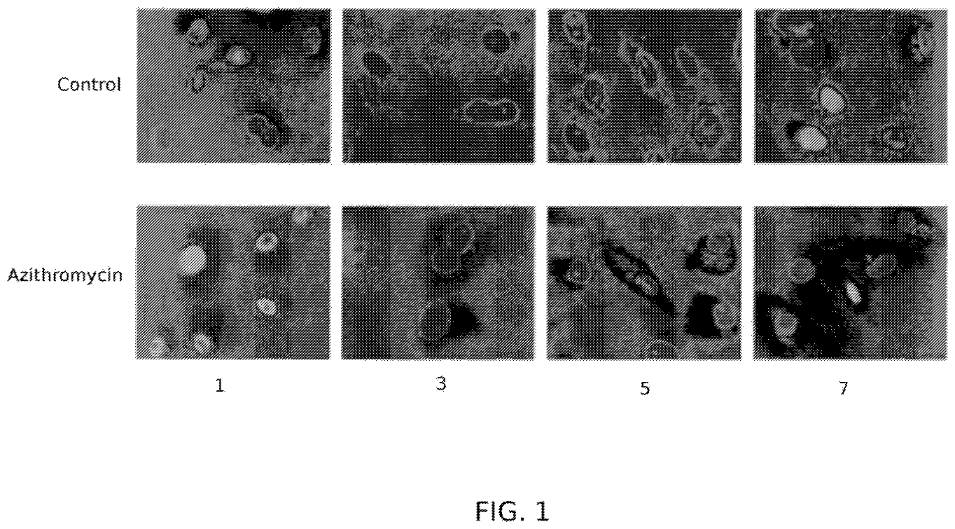

The results of these experiments are shown in FIGS. 1-5.

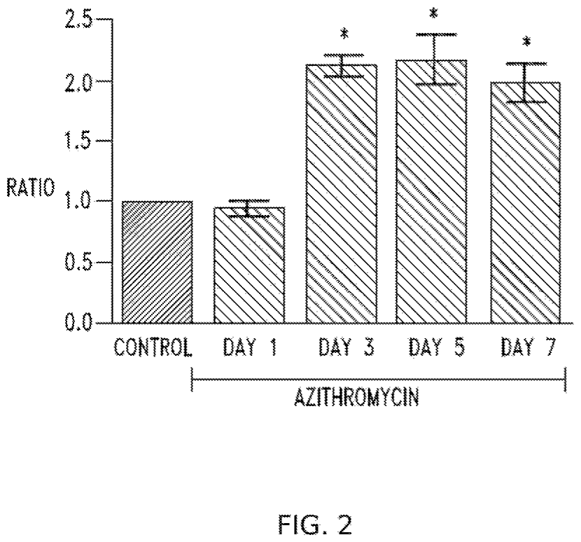

FIG. 1 shows that azithromycin induces a time-dependent accumulation of lipids in human meibomian gland epithelial cells. As shown in the figure, within 3 days of azithromycin exposure, the number, size and staining intensity of intracellular lipid-containing vesicles had markedly increased, as compared to those of vehicle-treated control cells. The azithromycin effect on lipids appeared to become maximal at days 3 to 7 as shown in FIG. 2.

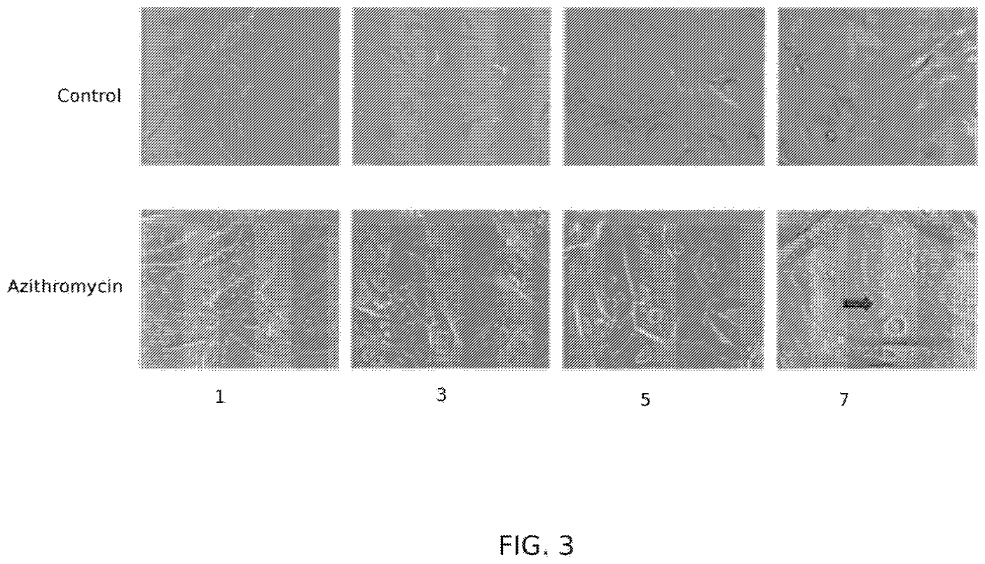

FIG. 3 shows that an evaluation of the cellular morphology indicated that the azithromycin may promote the terminal maturation of human meibomian gland epithelial cells, given that vesicle accumulation was often followed by a cell break-up and vesicle release.

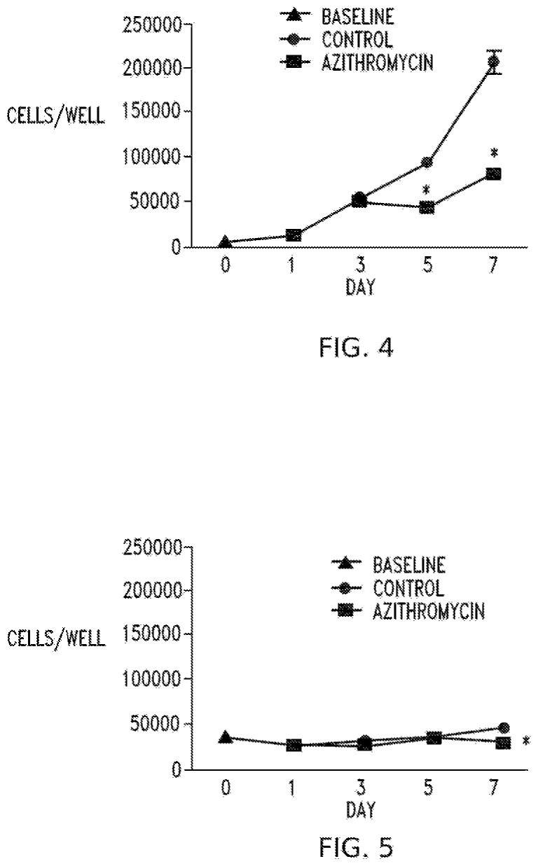

FIGS. 4 and 5 show that azithromycin generally reduced the proliferation of human meibomian gland epithelial cells, regardless of whether the cells were cultured under proliferation or differentiation conditions. FIGS. 4 and 5 were conducted in the absence and presence, respectively, of serum.

As shown in the examples above, azithromycin acts on human meibomian gland epithelial cells to stimulate lipid accumulation. This effect appears to be paralleled by cellular maturation, decreased cell proliferation, and a holocrine-like secretion.

### EXPERIMENTAL DATASET #1

Azithromycin can act directly on human meibomian gland epithelial cells to stimulate their differentiation, enhance the quality and quantity of lipid production, and promote holocrine secretion. Test data provided below supports this position.

Immortalized human meibomian gland epithelial cells (IHMGECs; passages 20-22) were cultured in the presence or absence of 10% fetal bovine serum. Cells were treated with the ethanol vehicle or azithromycin (10 μg/mL) for varying periods. Cellular morphological appearance was recorded, cells were counted with a hemocytometer, and lipid accumulation was assessed by staining cells with LipidTOX green neutral lipid stain (Invitrogen Corp). Staining fluorescent intensities were quantified using ImageJ software (http://rsbweb.nih.gov/ij/index.html). Statistical analyses were performed with t test (2-tailed, unpaired).

Experimental results show that azithromycin induces a striking, time-dependent accumulation of lipid in IHMGECs. Within 3 days of azithromycin exposure, the number, size, and staining intensity of intracellular lipid-containing vesicles had markedly increased as compared with those of vehicle-treated control cells. This azithromycin effect on lipids appeared to become maximal at days 3 to 7 of the study.

Evaluation of cellular morphology indicated that azithromycin may promote terminal maturation of IHMGECs given that vesicle accumulation was often followed by a cell break-up and vesicle release.

In contrast to these effects, azithromycin reduced the proliferation of IHMGECs. This result was found irrespective of whether IHMGECs were cultured under proliferation or differentiation conditions.

This experimental data demonstrates that azithromycin can act on human meibomian gland epithelial cells and stimulate their lipid accumulation. This azithromycin effect appears to be paralleled by a cellular maturation, a decreased proliferation, and a holocrine-like secretion.

This azithromycin action is quite notable because MGD is thought to be the most common cause of DED. Typically, the meibomian glands produce and release a lipid mixture that promotes the stability and prevents the evaporation of the tear film, thereby playing an essential role in ocular surface health. Conversely, MGD destabilizes the tear film and increases its evaporation. Meibomian gland dysfunction is caused primarily by hyperkeratinization of the terminal duct epithelium and reduced secretion quality, and it leads to cystic dilatation of glandular ducts, acinar cell death, and lipid deficiency. The end result is DED, characterized by a cycle of tear film hyperosmolarity and ocular surface stress and leading to increased friction, inflammation, and damage to the eye. The effect of moderate to severe DED is analogous to conditions such as dialysis and severe angina and is associated with significant pain, role limitations, low vitality, and poor general health.

The experimental data show that azithromycin stimulates the function and differentiation of IHMGECs in vitro, and that this antibiotic can be beneficial as a treatment for MGD and its associated DED in vivo.

### EXPERIMENTAL DATASET #2

Drug-induced phospholipidosis (PLD) is an excessive intracellular accumulation of phospholipid, characterized by the formation of distinct, onion-shaped secretory lysosomes, termed lamellar bodies. Drug-induced PLD can be caused by many drugs, especially cationic amphiphilic drugs (CADs). It is a major problem for the pharmaceutical industry because of potential toxicity and the huge expense to screen out PLD-inducing drugs each year. The development of some lead compounds has been terminated when PLD was seen in certain organs in clinical trials. The mechanism of PLD has been linked to enhanced cholesterol synthesis, but its significance to humans is unclear. Although it is generally considered as a “poisonous” effect to be eliminated, this lipid accumulation effect can be beneficial in the treatment of meibomian gland dysfunction (MGD). MGD is, as set forth above, the most common cause of dry eye disease (DED), which afflicts tens of millions of people in the United States, and is one of the leading reasons for patient visits to eye care practitioners, Meibomian glands normally produce abundant lipids (e.g. cholesterol and phospholipids), that accumulate in lysosomes, are secreted in a holocrine manner into lateral ducts, and ultimately released onto the ocular surface. These secretions enhance the stability and prevent the evaporation of the tear film, thereby playing a critical role in the well-being of the eye. However, MGD, and the associated lipid deficiency, disrupts this process, destabilizes the tear film, increases its evaporation and promotes DED. MGD also facilitates bacterial growth on the lid margin and inflammation in the adjacent conjunctiva (e.g. posterior blepharitis). Azithromycin (AZM), a potent PLD-inducing CAD, can elicit a PLD-like effect in human meibomian gland epithelial cells and serve as a treatment for MGD. More specifically, AZM can increase cholesterol and phospholipid levels, stimulate the formation of the lamellar bodies, and promote lipid accumulation in the lamellar lysosomes of these cells. The experimental data provided herein confirms these statements.

This section outlines the materials and methods that produced the experimental data. Immortalized human meibomian gland epithelial cells were cultured in the presence or absence of 10% fetal bovine serum. After reaching 80-90% confluence (˜5×106/well), cells were exposed to the vehicle (0.02% ethanol) or azithromycin (10 μg/ml) for 5 days and then processed for histological and biochemical procedures.

Cell numbers were enumerated with a hemocytometer. Cellular lipid and lysosome accumulation were examined by staining cells with: Filipin III (a fluorometric probe for identifying unesterified cholesterol), LipidTOX green neutral lipid stain, and LysoTracker® Red DND-99 (a fluorescent technique designed for labeling acidic organelles such as lysosomes). Lysosomal lamellar bodies were identified by transmission electron microscopy. In brief, after culture and treatment on 24 mm polycarbonate membrane transwell inserts (0.4 μm pore size), cells were fixed in a solution of 2% formaldehyde+2.5% glutaraldehyde, in 0.08 M sodium cacodylate buffer, pH 7.4, post-fixed with 2% osmium tetroxide in 0.1 M sodium cacodylate buffer, and stained en bloc with 2% aqueousuranyl acetate. Samples were then dehydrated in graded ethyl alcohol solutions and embedded by using the tEPON-812 epoxy resin kit. Cross sections (1 μM) were stained with 1% toluidine blue in 1% sodium tetraborate solution. Ultra-thin sections (60-90 nm) were imaged with a transmission electron microscope interfaced with a digital CCD camera. For biochemical assessments, cellular lipids were extracted with chloroform and methanol from samples with the same amount of protein. Lipids were then evaluated with high-performance thin-layer chromatography. With regard to the staining results, three Filipin, five LipidTox and four LysoTracker studies were conducted, and each experiment was performed in duplicate. Four random pictures were taken of each group, and four random areas were selected for the measurement of fluorescent intensities. The HPTLC experiments were performed in duplicate more than three times. Image data were quantified with ImageJ (http://rsbweb.nib.gov/ij/index.html). Electron microscopic analyses were performed in triplicate.

The experimental results demonstrate that AZM significantly stimulates the accumulation of free cholesterol, neutral lipids and lysosomes inhuman meibomian gland epithelial cells.

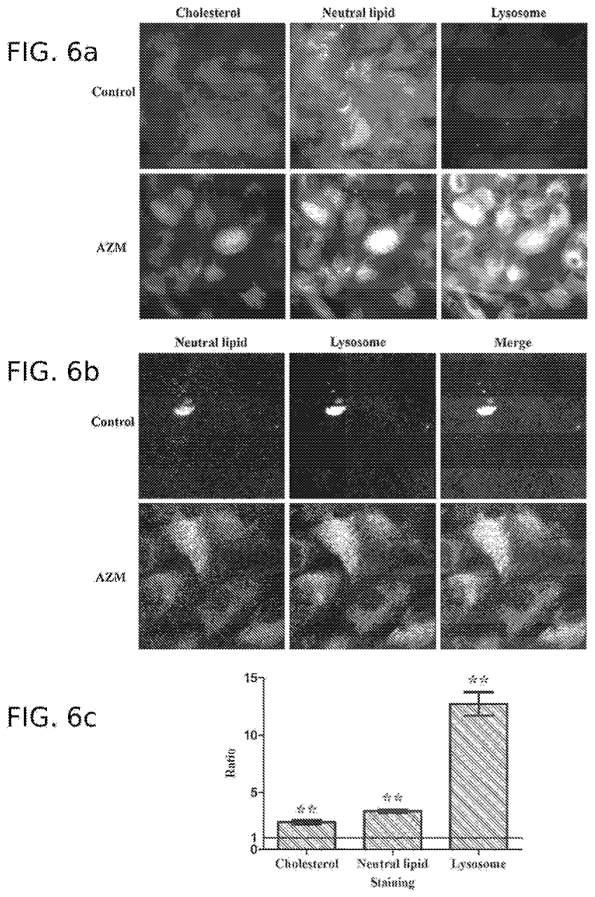

FIGS. 6a, 6b, and 6c illustrate the effect of AZM on intracellular accumulation of lipids and lysosomes. Cells were treated with vehicle or 10 μg/ml AZM for 5 days. FIG. 6a shows Filipin stain indicating free cholesterol, LipidTOX green neutral lipid stain, and LysoTracker red stain indicating lysosomes. Images were obtained with a fluorescent microscope. FIG. 6b shows cells stained as in FIG. 6a, and images were obtained with a confocal microscope in order to show colocalization of neutral lipids and lysosomes. FIG. 6c shows the fluorescence intensity measured using ImageJ. Control image intensity was set to 1 and data (mean±SE) are reported as fold-change compared to control values. Free cholesterol staining was repeated 3 times, neutral lipid staining 5 times and lysosome staining 4 times. The results shown are from a single experiment.

This AZM-induced increase of neutral lipid content occurred predominantly within lysosomes. Furthermore, electron microscopic analyses reveal that many of these lysosomes appear to be onion-shaped lamellar bodies.

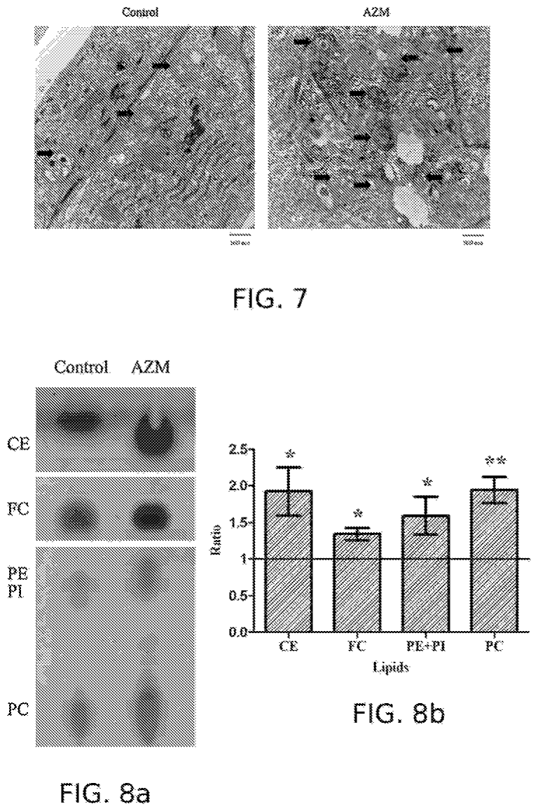

FIG. 7 illustrates the influence of AZM on the accumulation of lysosomal lamellar bodies in human meibomian gland epithelial cells. TEM images were obtained after cellular exposure to vehicle or 10 μg/ml AZM for 5 days. Arrows indicate the lamellar bodies. The bar shows scale at 500 nm.

Biochemical studies indicate that AZM significantly promotes an accumulation of free and esterified cholesterol, as well as phosphatidylethanolamine, phosphatidylcholine and phosphatidylinositol in human meibomian gland epithelial cells.

FIG. 8a illustrates the effect of AZM on the expression of cholesterol ester (CE), free cholesterol (FC), phosphatidylethanolamine (PE), phosphatidylcholine (PC), and phosphatidylinositol (PI) inhuman meibomian gland epithelial cells. Cells were treated with vehicle or 10 μg/ml AZM for 5 days before performing chromatographic analyses of total lipid extracts. FIG. 8b illustrates band intensity of the cells shown in FIG. 8a measured using ImageJ. Control band intensity was set to 1, and data (mean±SE) are reported as fold-change compared to control values. *p<0.05,**p<0.005, versus control. Band intensity analysis includes data from at least three independent experiments.

AZM induces a PLD-like effect in human meibomian gland epithelial cells. This macrolide antibiotic significantly stimulates the cellular accumulation of free and esterified cholesterol, neutral lipids, phospholipids and lysosomes. The increase of neutral lipid content occurred predominantly within lysosomes, many of which appeared to be lamellar bodies. Thus, topical AZM can be beneficial in the treatment of human MGD.

AZM can induce a PLD-like effect in human meibomian gland epithelial cells for several reasons. First, AZM promotes the accumulation of phospholipids, cholesterol and lysosomal lamellar bodies in other cells. One hallmark feature for identifying PLD is the demonstration of an intracellular accumulation of phospholipids and the concurrent development of lamellar bodies. A lamellar body is a type of lysosome specialized for lipid storage and secretion, and concentric vesicles like lamellar bodies appear to accumulate lipids in the meibomian gland. Second, AZM can act directly on human meibomian gland epithelial cells to seemingly stimulate their maturation and holocrine-like secretion. The effective concentration (i.e. 10 μg/ml) of AZM in vitro in the experimental data is clinically relevant. Following the topical application of 0.5, 1.0 and 1.5% AZM eye drops, the tear concentration of AZM remained above 7 μg/ml for 24 hours. Of particular note, the AZM-induced generation of lysosomes and the accumulation of lipids within these vesicles are analogous to events that typically occur during the differentiation of human meibomian gland epithelial cells. This cellular process is characterized by a pronounced increase in lysosome number and lipid production, and culminates with a profusion of lipid-filled vesicles and nuclear pyknosis. Following this terminal differentiation cells undergo holocrine secretion, which entails autophagy, apoptosis, disintegration and release of lipid-laden contents into glandular ductules. The ability of AZM to stimulate the differentiation, and apparently secretion, of human meibomian gland epithelial cells is clinically very significant. The experimental data indicate that AZM can directly enhance the function of human meibomian gland epithelial cells, and thereby ameliorate the pathophysiology of MGD.

The experimental data support the hypothesis that IGF-1 acts on human meibomian gland epithelial cells and may explain why treatment with figitumumab, the GF-1 inhibitor, causes dry eye disease. Ophthalmic care for dry eye disease may be needed when patients with cancer undergo treatment with drugs that inhibit IGF-1 action.

### EXPERIMENTAL DATASET #3

Hormone IGF-1 plays a very important role in human growth and development. Insulin-like growth factor 1 is a potent activator of the phosphoinositol 3-kinase (PI3K)/Akt pathway, which stimulates cell proliferation and differentiation and inhibits programmed cell death. The potency of IGF-1 action is such that alterations in its signaling pathway components may promote the development of a variety of malignant diseases. This recognition, in turn, has led to the generation of pharmaceuticals to block the IGF-1 receptor (IGF-1R) and serve as anticancer treatments.

One such drug is figitumumab, a human IgG2 monoclonal antibody that prevents the binding of IGF-1 to its receptor, blocks IGF-1 downstream signaling, and induces IGF-1R degradation. Of particular interest, the most common adverse effect of figitumumab in a one trial was DED. DED is one of the most frequent causes of patient visits to eye care practitioners and affects tens of millions of people in the United States. This condition is characterized by a vicious cycle of tear film hyperosmolarity and ocular surface stress, leading to increased friction, inflammation, and damage to the eye. The impact of moderate to severe DED is comparable to that of conditions such as dialysis and severe angina and is associated with significant pain and role limitations, low vitality, and poor general health.

The experimental data show that the mechanism by which figitumumab induces DED is inhibition of IGF-1 action in the epithelial cells of the meibomian glands. These large sebaceous glands typically secrete lipids that increase the stability and decrease the evaporation of the tear film, thereby promoting the health and well-being of the ocular surface. However, their dysfunction, termed meibomian gland dysfunction (MGD), leads to lipid insufficiency, tear film hyperosmolarity and instability, and evaporative DED. Meibomian gland dysfunction is the primary cause of DED worldwide.

In support of this hypothesis, investigators have reported that IGF-1 stimulates the function of epithelial cells in other sebaceous glands. More specifically, IGF-1 activates the PI3K/Akt pathway, enhances proliferation, and augments lipid biosynthesis in rat and/or human sebaceous gland cells. The lipid effect appears to be linked to an upregulation of sterol regulatory element-binding protein 1 (SREBP-1), a key transcription factor that impels lipogenesis. However, given that considerable differences exist in the control of sebaceous glands between species and between different types of sebaceous glands, 23 IGF-1 may or may not exert similar actions in the epithelial cells of human meibomian glands.

The experimental data demonstrate that IGF-1 acts on human meibomian gland epithelial cells, and that IGF-1 activates the PI3K/Akt pathway, stimulates proliferation, increases SREBP-1 expression, and promotes lipid accumulation in these cells. IGF-1 (1) can activate the extracellular signal-regulated kinase (i.e., ERK; also known as mitogen-activated protein kinase) pathway, which is involved in IGF-1 signaling in other cell types; (2) modulates the phosphorylation of forkhead box O1 (FoxO1), a transcription factor that, when phosphorylated by Akt, is inhibited from reducing cell proliferation, SREBP-1 expression, and lipogenesis; and (3) elicits effects analogous to those of growth hormone (GH). GH and IGF-1 act in concert to influence sebaceous gland function and dysfunction. GH signaling mainly involves the Janus kinase 2 (JAK2)/signal transducers and activators of transcription 5 (STAT5) pathway but also may include the ERK and PI3K/Akt pathways.

The disclosure turns to the methods used to generate the experimental data. First the methods relate to cell culture and treatment. Immortalized human meibomian gland epithelial cells were maintained in keratinocyte serum-free medium supplemented with 5 ng/mL of epidermal growth factor (EGF) and 50 μg/mL of bovine pituitary extract (BPE). When indicated, cells were cultured in basal keratinocyte serum-free medium alone or a supplemented serum-free medium supplemented with 1% or 10% fetal bovine serum (FBS).

The EGF- BPE- and 10% FBS-containing media can promote epithelial cell proliferation and differentiation, respectively, in human meibomian gland epithelial cells. Recombinant human IGF-1 and human GH were dissolved in phosphate-buffered saline, and filter sterilized. The IGF-1 and GH were applied at a final concentration of 10 nM to cells.

The disclosure turns now to sodium dodecyl sulfate-polyacrylamide gel, electrophoresis, and immunoblots. Cells were directly lysed in Laemmli buffer supplemented with 1% protease inhibitor cocktail (Sigma-Aldrich) and 5% β-mercaptoethanol (Sigma-Aldrich). Lysates were heated at 95° C. for 10 minutes, separated by sodium dodecyl sulfate-polyacrylamide gel electrophoresis on 10% Tris-glycine precast gels, and transferred to polyvinylidene difluoride membranes. Rabbit or mouse antibodies used were specific for phosphorylated Akt (p-Akt), pan-Akt, p-FoxO1, FoxO1, p-JAK2, JAK2, and β-actin; for p-ERK, ERK-2, STAT5, and the precursor form of SREBP-1; and for p-STAT5. Membranes were blocked with 5% bovine serum albumin in Tris-buffered saline containing 0.01% polysorbate 20. All primary antibodies were diluted to 1:1000 in blocking buffer except for p-Akt (1:4000), p-ERK (1:2000), ERK-2 (1:4000), SREBP-1 (1:200), and β-actin (1:10 000). Horseradish peroxidase-conjugated secondary antibodies were goat anti-rabbit IgG and Fc-specific goat anti-mouse IgG diluted to 1:5000. Proteins were visualized with enhanced chemiluminescent substrate or with ultrasensitive chemiluminescent substrate. Densitometry was performed with image-processing software.

Regarding cell proliferation assay, the human meibomian gland epithelial cells and LNCaP cells were cultured in 12-well plates in designated media and then exposed to IGF-1 or GH for defined periods. At experimental termination, cells were trypsinized and counted with a hemocytometer.

The experiment included green neutral lipid staining. Immortalized human meibomian gland epithelial cells were cultured in slides treated to facilitate cell growth in FBS-containing medium in the presence or absence of IGF-1, anti-IGF-1R, and/or GH. Media were replaced every 2 to 3 days for a total of 6 days, at which time cells were washed and fixed in 4% paraformaldehyde for 30 minutes. After additional washes, cells were stained with green neutral lipid stain in a humid chamber for 30 minutes. Coverslips were mounted on slides with an antifade reagent that contains 4′,6-diamidino-2-phenylindole nuclear stain and permitted to dry overnight before imaging with a microscope. Fluorescence intensity was quantified using image-processing software.

The experimental results show the influence of IGF-1 and GH on signaling pathways. To determine whether IGF-1 promotes the phosphorylation of Akt in human meibomian gland epithelial cells, cells were cultured under proliferating or differentiating conditions and then exposed to IGF-1 for 15 minutes.

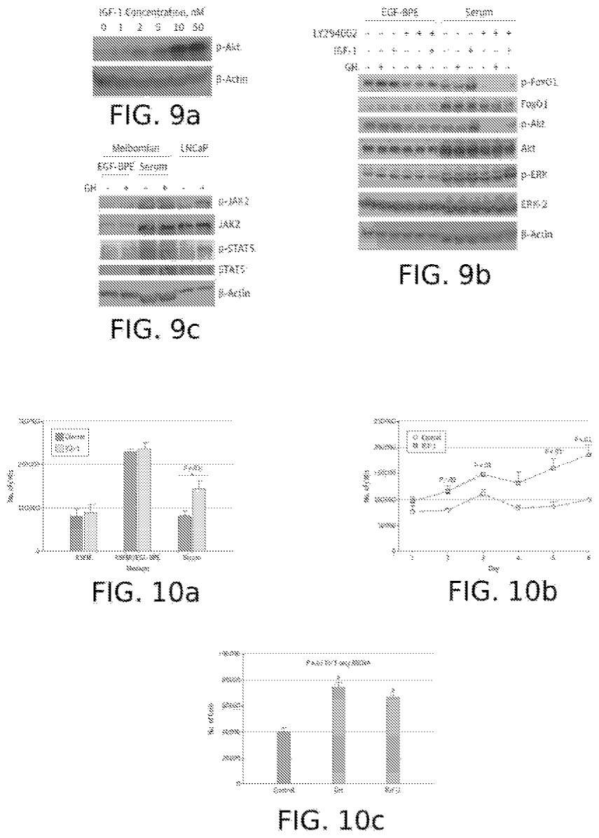

FIGS. 9a, 9b, and 9c illustrate the effects of insulin-like growth factor 1 (IGF-1) and growth hormone (GH) on signaling pathways in human meibomian gland epithelial cells. Cells were cultured in keratinocyte serum-free medium containing epidermal growth factor (EGF) and bovine pituitary extract (BPE) for 2 days and then keratinocyte serum-free medium alone overnight or were cultured in 10% fetal bovine serum (FBS) medium for 5 to 7 days and in 1% FBS medium overnight. Cells were then incubated with vehicle or a 10 μM concentration of LY294002 for 2 hours, followed by IGF-1 or GH treatment for 15 minutes. Cell lysates were evaluated on immunoblotting. FIG. 9a shows a dose-dependent effect of IGF-1 on Akt phosphorylation. FIG. 9b shows IGF-1 and GH influence on the phosphorylated and total levels of Akt, forkhead box O1 (FoxO1), and extracellular signal-regulated kinase (ERK). Minus signs indicate “without.” Plus signs indicate “with.” FIG. 9c shows the effect of GH on the Janus kinase 2/signal transducers and activators of transcription 5 (JAK2/STAT5) pathways in meibomian gland epithelial cells and the LNCaP cancer cell line. These experiments were repeated at least 3 times with similar results.

IGF-1 induced a dose-dependent expression of p-Akt. This effect could be detected after exposure to a 1 nM concentration of IGF-1 and became maximal at a 10 nM concentration. This latter dose was used for all other studies in this section. The stimulatory influence of IGF-1 on Akt was paralleled by an increased phosphorylation of FoxO1, and these actions occurred in differentiating but not proliferating cells. These differentiated effects were blocked by preincubation with the PI3K inhibitor LY294002. In contrast, IGF-1 had an inconsistent effect on ERK. In 2 studies with differentiating cells, IGF-1 enhanced the phosphorylation of ERK, whereas in 3 other experiments, IGF-1 had no activating influence on ERK.

To assess whether GH activates Akt, FoxO1, ERK-2, or its classic (i.e., JAK2 and STAT5) signaling pathways in human meibomian gland epithelial cells, cells were cultured in proliferating or differentiating media, and were exposed to a 10 nM concentration of GH for 15 minutes, and processed for immunoblot analyses. The experimental data did not show that GH elicited effects on these signaling components. The same negative results were also found at GH doses of 0.05 to 50 nM. For comparison, GH activated the JAK2/STAT5 pathway in the positive control LNCaP cells.

To evaluate whether IGF-1 stimulates proliferation of human meibomian gland epithelial cells, cells were cultured in basal, proliferating, or differentiating medium and treated with IGF-1 for 2 days.

FIGS. 10a, 10b, and 10c illustrate the influence of IGF-1 on the proliferation of human meibomian gland epithelial cells. Cells were cultured (100,000 cells/well in 12-well plates, 3 wells per group) in basal, proliferating, and/or differentiating media and treated with IGF-I for 2 to 6 days. FIG. 10a shows meibomian gland epithelial cell proliferation. FIG. 10b shows meibomian gland cell proliferation in serum-containing media for 6 days. P values are for post hoc comparison compared with the control value. FIG. 10c shows LNCaP cells (20,000 cells/well in 12-well plates, 3 wells per group) were initially cultured in Dulbecco modified Eagle medium/Ham F-12 nutrient media mixture containing 10% fetal bovine serum (FBS), then switched to 1% FBS and exposed to IGF-1 or growth hormone (GH) for 2 days. ANOVA indicates analysis of variance; EGF-BPE, epidermal growth factor-bovine pituitary extract; KSFM, keratinocyte serum-free medium. All comparisons are with control value. Whiskers mark SEM. aP<0.05 compared with the control value, post hoc test.

IGF-1 induced an increase in cell number in the 10% FBS-containing medium but not under basal conditions (i.e., keratinocyte serum-free medium) or in media already primed to promote cellular proliferation (i.e., keratinocyte serum-free medium and the EGF-BPE mixture). This stimulatory effect of IGF-1 on cell proliferation continued throughout a 6-day course. In contrast, GH had no influence on the proliferation of human meibomian gland epithelial cells in any of the media at doses ranging from 0.1 to 100 nM. The proliferation of LNCaP control cells was increased by IGF-1 and GH.

Turning to the influence of IGF-1 and GH on SREBP-1 expression and lipid accumulation, the experiments examined whether IGF-1 stimulates SREBP-1 expression and lipid accumulation in human meibomian gland epithelial cells. Cells were cultured for 6 days in differentiation media with IGF-1 and then processed the cells for protein and histologic analysis. IGF-1 induced a rise in the amount of the precursor form of SREBP-1 protein. This stimulatory effect was accompanied by an increase in the accumulation of neutral lipids. These IGF-1 actions were not duplicated by cellular treatment with GH.

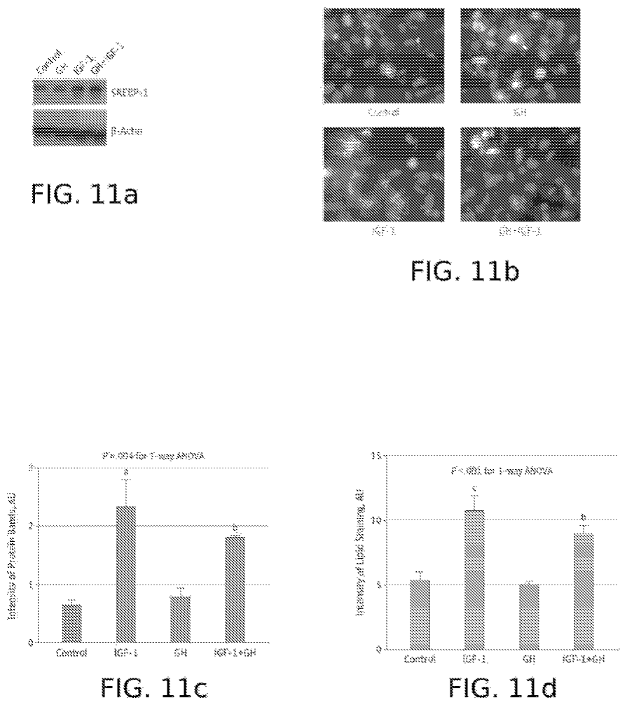

FIGS. 11a, 11b, 11c, and 11d illustrate the effect of IGF-1 on sterol regulatory element-binding protein (SREBP-1) expression and lipid accumulation in human meibomian gland epithelial cells. Cells were exposed to IGF-1 or growth hormone (GH) for 6 days in differentiation media and then processed for the analysis of the precursor form of SREBP-1 protein. FIG. 11a shows results of immunoblotting. FIG. 11b shows results of green neutral lipid staining. The red color represents 4′,6-diamidino-2-phenylindole nuclear staining. The SREBP-1 densitometry, normalized to that of β-actin, and neutral lipid staining fluorescence intensity (6 field views per treatment condition) were quantified by using image-processing software. These experiments were repeated at least 3 times with similar results. ANOVA indicates analysis of variance; AU, arbitrary units. Whiskers represent SEM.

To determine whether anti-IGF-1R is able to inhibit IGF-1 action on human meibomian gland epithelial cells, cells were cultured in differentiating conditions and exposed to anti-IGF-1R for varying periods.

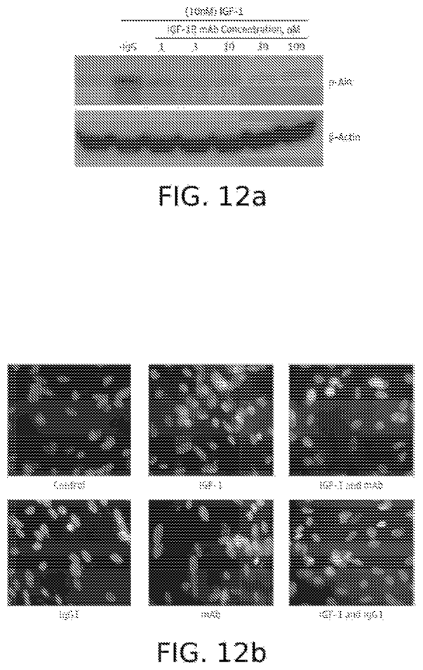

FIGS. 12a and 12b illustrate the inhibition of IGF-1 action by an IGF-1 receptor (IGF-1R)-blocking antibody in human meibomian gland epithelial cells. FIG. 12a shows cells that were cultured in medium containing 10% fetal bovine serum (FBS) for 5 days and then switched to 1% FBS medium containing various doses of anti-IGF-1R overnight, followed by IGF-1 treatment for 15 minutes. Antibody treatment inhibited the IGF-1-induced Akt phosphorylation in a dose-dependent manner. FIG. 12b shows cells that were exposed to a 10 nM concentration of IGF-1 and/or a 10 nM concentration of anti-IGF-1R for 6 days in serum-containing media and then processed for green neutral lipid staining. The red color represents 4′,6-diamidino-2-phenylindole nuclear staining. The antibody reduced the IGF-1-stimulated accumulation of lipids. These experiments were repeated twice with similar results. mAb indicates monoclonal antibody.

Anti-IGF-1R suppressed the ability of IGF-1 to phosphorylate Akt. This inhibitory effect of anti-IGF-1R was found at a 1 nM dose, became maximal at 10 nM, and could not be duplicated by the use of an irrelevant IgG antibody. In addition to this interference with IGF-1-mediated signaling, the anti-IGF-1R also specifically blocked the IGF-1-linked accumulation of lipids in human meibomian gland epithelial cells.

This experimental data demonstrates that IGF-1 exerts a marked influence on the function of human meibomian gland epithelial cells. IGF-1 activates the PI3K/Akt and FoxO1 pathways, stimulates proliferation, increases SREBP-1 expression, and promotes lipid accumulation in these cells. These IGF-1 actions are not accompanied by consistent effects on ERK phosphorylation and are not duplicated by GH. Further, anti-IGF-1R blocks the cellular signaling and lipid accumulation induced by IGF-1, demonstrating that IGF-1 exerts its action via IGF-1R. The experimental data shows that IGF-1 acts on human meibomian gland epithelial cells and may explain why treatment with figitumumab, the IGF-1 inhibitor, causes DED.

The stimulation of cell proliferation, SREBP-1 expression, and lipid production by IGF-1 may be mediated through its activation of the PI3K/Akt and FoxO1 pathways. Further, IGF-1 increased the levels of p-FoxO1, which prevents the FoxO1 suppression of cell proliferation and lipogenesis. It is likely that PI3K/Akt is the intermediate of the IGF-1 effect on FoxO1, given that IGF-1 action was considerably reduced in the presence of the PI3K inhibitor LY194002.

IGF-1 can stimulate cell proliferation in media containing 10% FBS but not EGF-BPE. The FBS-containing medium, which primes human meibomian gland epithelial cells for differentiated functions, can support IGF-1-associated proliferation. However, EFG-BPE-containing medium promotes the proliferation of human meibomian gland epithelial cells, a process involving a significant increase in the expression of cell cycle genes (e.g., cyclins B2, D1, D2, and D3; cyclin-dependent kinases 4 and 6; and E2F transcription factor). Given that IGF-1 also upregulates cyclins D1 and D3 to stimulate cell proliferation, this hormonal effect may be undetectable because before IGF-1 treatment, EGF and BPE have already maximally stimulated the major molecular components driving cell cycle progression.

The experimental data indicate that IGF-1 inconsistently activates ERK and that GH does not elicit detectable effects in human meibomian gland epithelial cells. The lack of a consistent IGF-1 activation of ERK suggests that this pathway is not the major one for IGF-1 in these cells. The experimental data does not indicate activations of Akt, FoxO1, ERK, JAK2, and/or STAT5 or changes in cell proliferation or lipid accumulation by GH treatment, suggesting that these signaling systems and responses may not be susceptible to GH influence in human meibomian gland epithelial cells. GH increases the percentage of lipid-forming colonies of rat preputial sebaceous gland cells, but this effect is relatively small in the absence of exogenous insulin. GH does not modulate DNA synthesis in these preputial cells.

The mechanism by which figitumumab induces DED is inhibition of IGF-1 action in human meibomian gland epithelial cells. Such an inhibition could reduce glandular lipid accumulation, lead to a lipid insufficiency on the ocular surface, and ultimately cause evaporative DED. Another possible consequence of IGF-1 blockade might be a loss of androgen influence in this tissue. A decrease in downstream IGF-1 receptor signaling can attenuate the expression and function of androgen receptors in human prostatic cells. Androgen receptor dysfunction, in turn, is a significant risk factor for the development of meibomian gland dysfunction and DED.

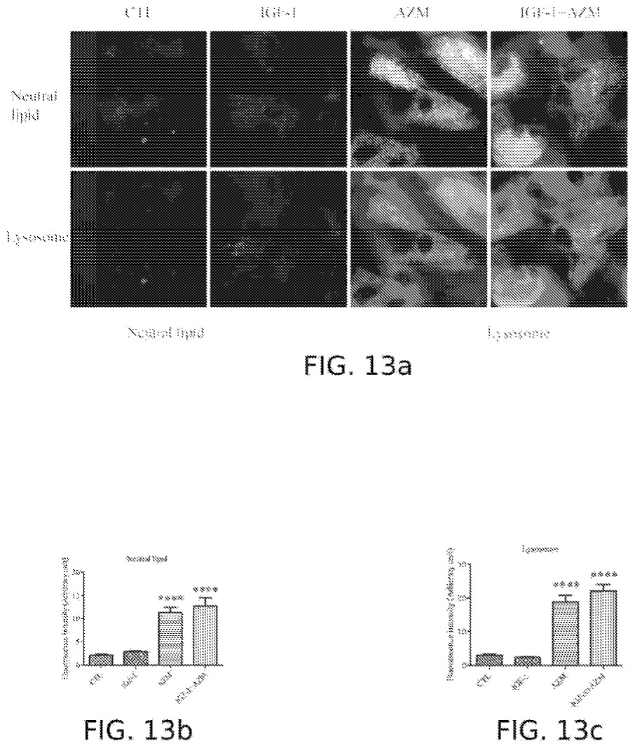

FIGS. 13a, 13b, and 13c illustrate the effect of IGF-1, AZM, and IFG-1+AZM combination on intracellular accumulation of lipids and lysosomes. Cells were treated with vehicle, 10 nM IGF-1, 10 μg/mL AZM, or IGF-1+AZM combination for 13 days. FIG. 13a represents LipidTOX green neutral lipid staining, and red color LysoTracker staining for lysosomes. FIG. 13b quantifies the fluorescence intensity of LipidTOX staining using ImageJ. Two-way ANOVA showed significant effect of AZM (****P<0.0001). FIG. 13c quantifies the fluorescence intensity of LysoTracker staining using ImageJ. Two-way ANOVA showed significant effect of AZM (P<0.0001). The experiments were repeated four times with similar results; data shown here are from a single experiment.

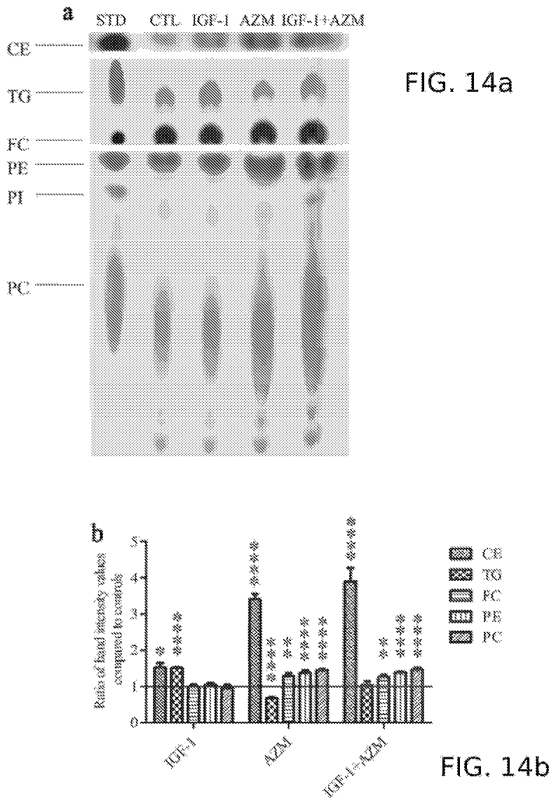

FIGS. 14a and 14b illustrate the effect of IGF-1, AZM, and IFG-1+AZM combination on the accumulation of CE, TG, FC, PE, and PC. FIG. 14a shows cells treated with vehicle, 10 nM IGF-1, 10 μg/mL AZM, or IGF-1+AZM combination for 7 days before performing chromatographic analyses of total lipid extracts. FIG. 14b quantifies band intensity using ImageJ. The control band intensity was set to 1, and data (mean 6 SE) were reported as fold-change compared with control values. The IGF-1 showed a significant effect on CE (*P<0.05) and TG (****P<0.0001). The AZM showed a significant effect on CE, TG, PE, PC (P<0.0001 for all four), and FC (**P<0.01). Other bands are unidentified lipids. Band intensity analysis included data from three independent experiments.

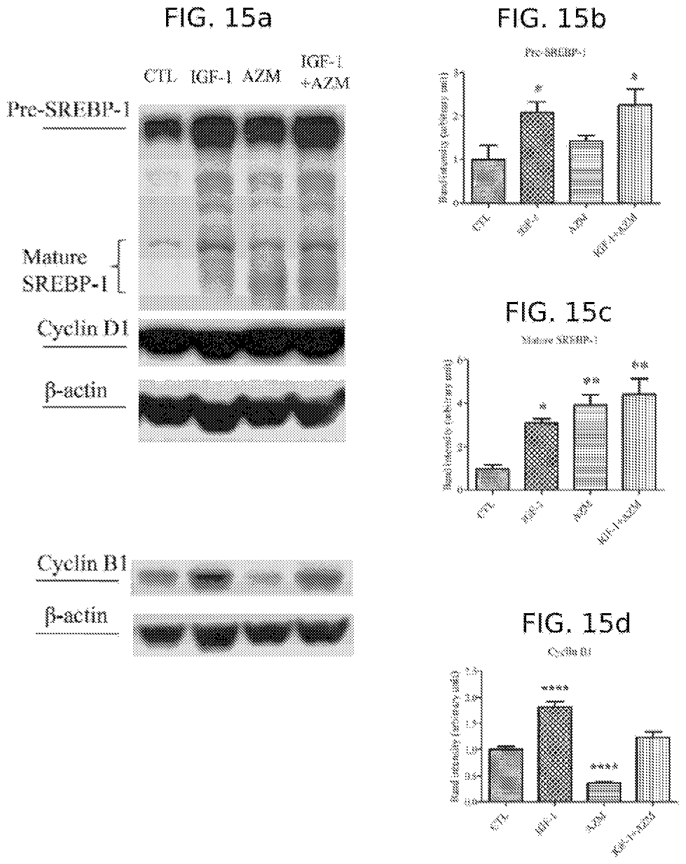

FIGS. 15a, 15b, 15c, and 15d illustrate the effect of IGF-1, AZM, and IFG-1+AZM combination on the expression of SREBP-1, cyclins B1, and D1. Cells were incubated with vehicle, 10 nM IGF-1, 10 μg/mL AZM, or IGF-1+AZM combination for 5 days. Cell lysates were evaluated on immunoblots for precursor and mature forms of SREBP-1, cyclins B1 and D1. FIG. 15a quantifies the protein band intensities using ImageJ. FIGS. 15b, 15c, and 15d illustrate how the IGF-1 significantly affected the expression of pre-SREBP-1 and mature SREBP-1 (*P<0.05) and cyclin B1 (****P<0.0001). The AZM showed significant effect on mature SREBP-1 (**P<0.01) and cyclin B1 (P<0.0001). These experiments were repeated at least three times with similar results.

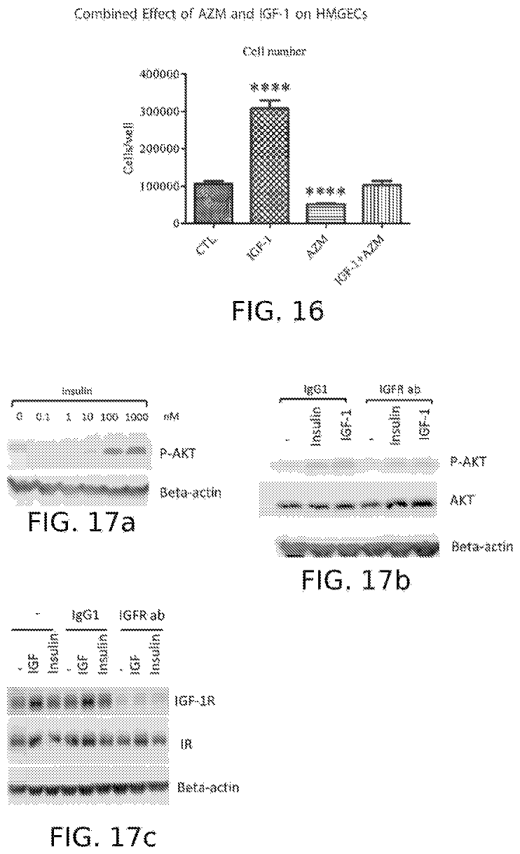

FIG. 16 illustrates the effect of IGF-1, AZM, and IFG-1+AZM combination on the proliferation of IHMGECs. Cells were seeded (50,000 cells/well in 12-well plates, n=3 wells/group) and treated with vehicle, 10 nM IGF-1, 10 μg/mL AZM, or IGF-1+AZM combination for 13 days before cell counting. Results were reported as mean±SE. The IGF-1 and AZM both exerted a significant but opposite effect on cell proliferation (****P<0.0001 for both). Data from one experiment were shown as a representative of three studies performed under the same conditions.

### Insulin

Insulin, an IGF-1 analogue, acts on the human meibomian gland epithelial cells similarly with IGF-1. For example, insulin activates the same Akt signaling pathway, and this activation is blocked by blocking the IGF-1 receptor. Notably, this blocking of IGF-1 receptor does not affect the insulin receptor, demonstrating insulin is indeed acting via IGF-1 receptor. In addition, insulin promotes meibomian gland epithelial cell proliferation, just like IGF-1. Further, insulin also promotes meibomian gland epithelial cell accumulation of neutral lipids, also similar to IGF-1. Therefore insulin, as an analogue of IGF-1, can stimulate meibomian gland function by activating AKT signaling, promoting proliferation and lipid accumulation in meibomian gland epithelial cells. Insulin activates AKT signaling pathway in a dose-dependent manner. Insulin-activation of AKT is similarly regulated by an IGF-1 receptor blocking antibody (insulin=200 nM, and IGF-1=10 nM). IGF-1 receptor antibody diminishes the IGF-1 receptor, but does not affect the insulin receptor (insulin=200 nM, and IGF-1=10 nM). Immortalized human meibomian gland epithelial cells were cultured in serum-containing medium for 5-7 days before treatment of insulin or IGF-1.

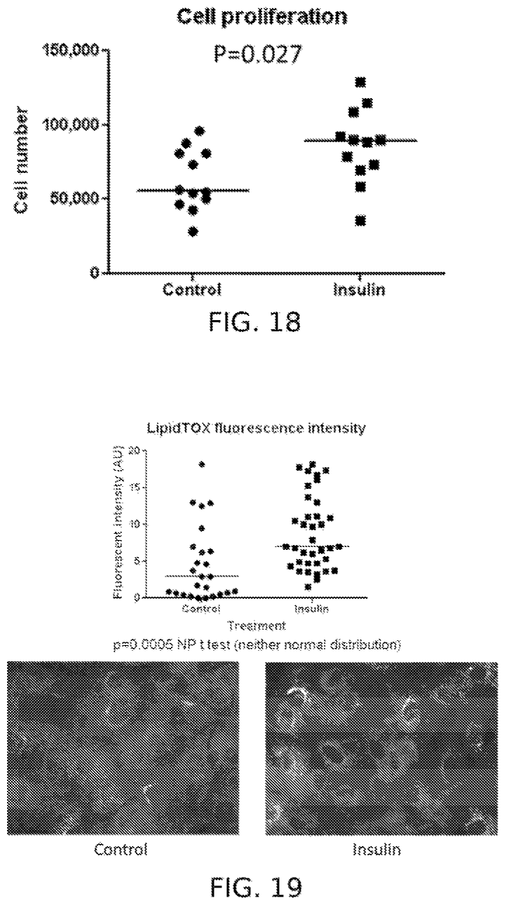

FIG. 17a illustrates how insulin activates AKT signaling in a dose-dependent manner. FIG. 17b illustrates that insulin activation of AKT is similarly regulated by an IGF-1 receptor blocking antibody (insulin=200 nM, and IGF-1=10 nM). FIG. 17c illustrates that IGF-1 receptor antibody diminishes the IGF-1 receptor without affecting the insulin receptor (insulin=200 nM, and IGF-1=10 nM). Immortalized human meibomian gland epithelial cells were cultured in serum-containing medium for 5-7 days before treatment of insulin or IGF-1. FIG. 18 illustrates that insulin promotes human meibomian gland epithelial cell proliferation. FIG. 19 illustrates that insulin promotes human meibomian gland epithelial cell accumulation of neutral lipids.

### Growth Hormone and IGF-1 Correlates to Meibomian Gland Size in Mice

GH and IGF-1 activity is positively correlated with meibomian gland size in mice. The (GH)/IGF-1 axis positively regulates meibomian gland size. To test this hypothesis, both upper and lower eyelids were dissected containing meibomian glands from bovine (b) GH transgenic mice (bGH mice), GH receptor knockout (GHR-/-) mice, GH antagonist (GHA) transgenic mice and their wild type (WT) littermate controls. Their meibomian gland size was compared using hematoxylin and eosin stained tissue slides. These animals represent mice with excess GH/IGF-1 signaling (bGH mice), absence of GH signaling and low IGF-1 signaling (GHR-/- mice), and deficient GH/IGF-1 signaling (GHA mice).

Significantly increased meibomian gland size was shown in bGH compared to WT mice in both upper and lower eyelids. The mean increase is over 2 fold for both upper and lower lid meibomian glands. GHR-/- mice, on the other hand, showed significantly smaller meibomian glands in both upper and low lids, with mean value 36% and 41% that of the WT control for upper and lower lid meibomian glands, respectively. The GHA mice show significantly smaller upper lid meibomian glands, but no significant difference in the lower lid. The meibomian gland size of GHA mice relative to WT control mice is 58% and 82% for upper and lower lid, respectively.

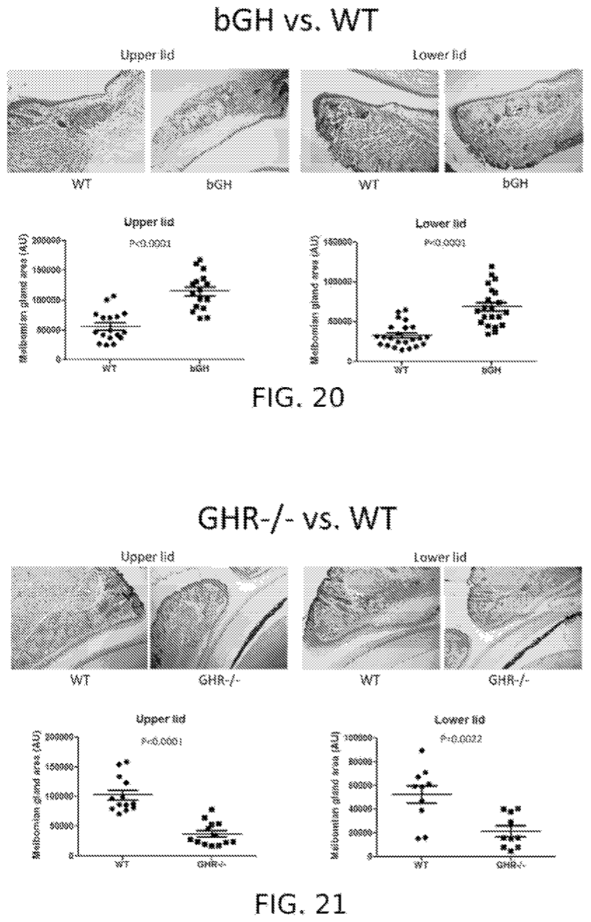

FIG. 20 illustrates increased meibomian gland size in bGH mice that overexpress bovine growth hormone compared to wild type (WT) mice. The upper and lower lid tissue are stained to show the meibomian gland, and the charts show quantification of upper and lower meibomian gland size.

FIG. 21 illustrates decreased meibomian gland size in GHR-/- mice that have no growth hormone receptors compared to WT control mice. The upper and lower lid tissue are stained to show the meibomian gland, and the charts show quantification of upper and lower meibomian gland size.

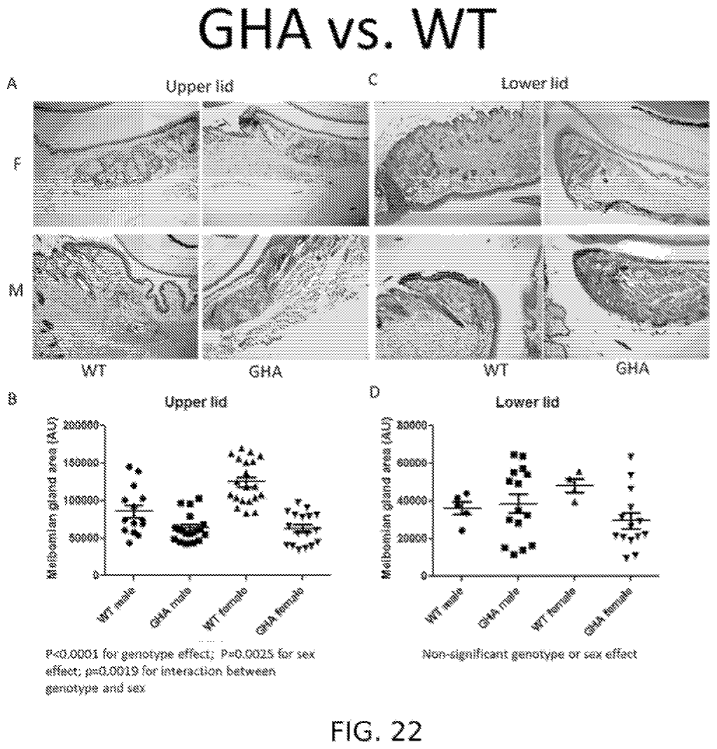

FIG. 22 illustrates decreased meibomian gland size in GHA mice which have growth hormone deficiency compared to the WT control mice. The upper and lower lid tissue are stained to show the meibomian gland, and the charts show quantification of upper and lower meibomian gland size.

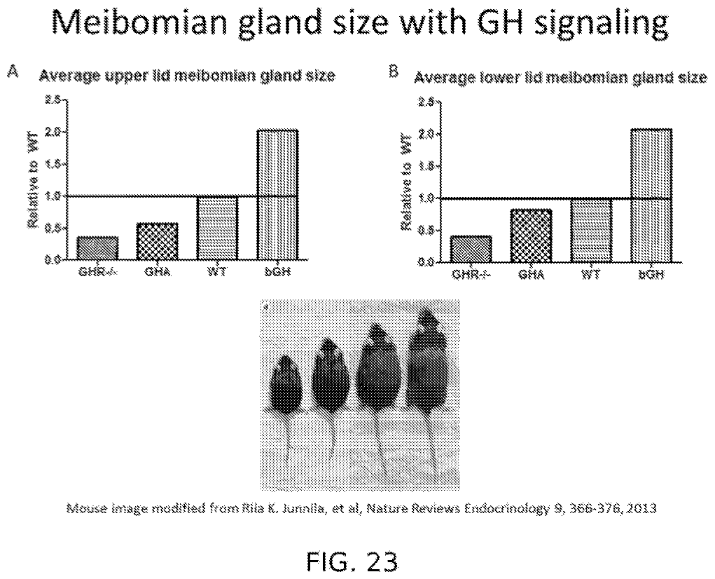

FIG. 23 illustrates relative meibomian gland size when normalized to the WT controls for GHR-/- (no GH signaling), GHA (GH deficiency), and bGH (GH excess) mice.

From the foregoing, it will be appreciated that, although specific embodiments have been described herein for purposes of illustration, various modifications may be made without deviating from the spirit and scope of the disclosure as set forth in the appended claims. All publications, patents, and patent applications referenced herein are incorporated by reference in their entirety.

1.-16. (canceled)

17. A method for stimulating of human meibomian gland epithelial cell function, the method comprising: preparing a topical formulation of a pharmaceutical composition comprising a therapeutic agent of between and including 0.1% and 2%, by mass/volume, of a PLD-inducing compound, wherein the PLD-inducing compound comprises a cationic amphiphilic drug; and administering the topical formulation to an ocular surface of a subject or to an area immediately adjacent thereto.

18. The method of claim 17, wherein the topical formulation is applied to the ocular surface of a subject.

19. The method of claim 17, wherein the topical formulation is applied to a region of an eye immediately adjacent to the ocular surface.

20. The method of claim 17, wherein the pharmaceutical composition further comprises hyaluronate.

21. The method of claim 17, wherein the pharmaceutical composition further comprises at least one electrolyte selected from the group consisting of sodium chloride, potassium chloride, sodium bicarbonate, potassium bicarbonate, calcium chloride, magnesium chloride, trisodium citrate, hydrochloric acid, sodium hydroxide, and mixtures thereof.

22. The method of claim 17, wherein the pharmaceutical composition further comprises at least one additive selected from the group consisting of ophthalmic demulcents, excipients, astringents, vasoconstrictors and emollients.

23. A method of stimulating human meibomian gland epithelial cell function, comprising
identifying a subject comprising an eye disorder of the ocular surface, said eye disorder comprising a meibomian gland dysfunction (MGD), evaporative Dry Eye Disease (DED), a lipid abnormality of meibum or the tear film, or Sjogren's syndrome and
topically administering to an eye surface of said subject a pharmaceutical composition comprising a cationic amphiphilic drug (CAD), wherein said CAD comprises azithromycin in an amount to elicit a Drug-induced phospholipidosis (PLD)-like effect in said eye, wherein said amount is 2% by mass/volume or less of said azithromycin.

24. The method of claim 23, wherein said azithromycin enhances the quality and quantity of lipid production and promotes holocrine secretion thereof, thereby ameliorating the pathophysiology of MGD.

25. The method of claim 23, wherein said composition comprises an eye drop.

26. The method of claim 25, wherein said eye drop comprise 0.1-2.0% of said azithromycin.

27. The method of claim 25, wherein said eye drop comprise 0.5-2.0% of said azithromycin.

28. The method of claim 25, wherein said eye drop comprise 0.5-1.5% of said azithromycin.

29. The method of claim 25, wherein said eye drop comprise 0.5% of said azithromycin.

30. The method of claim 25, wherein said eye drop comprise 1.0% of said azithromycin.

31. The method of claim 25, wherein said eye drop comprise 1.5% of said azithromycin.

32. The method of claim 23, wherein said pharmaceutical composition further comprises a hyaluronate, an electrolyte, an ophthalmic demulcent, an excipient, an astringent, a vasoconstrictor, or an emollient.

33. The method of claim 23, wherein the pharmaceutical composition further comprises at least one electrolyte selected from the group consisting of sodium chloride, potassium chloride, sodium bicarbonate, potassium bicarbonate, calcium chloride, magnesium chloride, trisodium citrate, hydrochloric acid, and sodium hydroxide.

34. The method of claim 23, wherein said human meibomian epithelial cell function comprises an intracellular accumulation of lipids, an intracellular accumulation of lysosomes, increased cholesterol, increased phospholipid levels, formation of lamellar bodies, or lipid accumulation in said lysosomes.

35. The method of claim 23, wherein said human meibomian epithelial cell function comprises an intracellular accumulation of lipids

36. A method of directly stimulating human meibomian gland epithelial cell function, comprising administering to meibomian gland epithelial cells a pharmaceutical composition comprising a PLD-inducing cationic amphiphilic drug, wherein said drug enhances lipid production and promotes holocrine secretion thereof.

37. The method of claim 36, wherein said drug comprises azithromycin at a concentration of 2% (mass/volume) or less.
